(19) United States
(12) Patent Application Publication
(10) Pub. No.: US 2017/0181711 A1
(43) Pub. Date: Jun. 29, 2017

(54) TIME-VARYING RISK PROFILING FROM HEALTH SENSOR DATA

(71) Applicant: INTERNATIONAL BUSINESS MACHINES CORPORATION, ARMONK, NY (US)

(72) Inventors: YU CHENG, YORKTOWN HEIGHTS, NY (US); JIANYING HU, YORKTOWN HEIGHTS, NY (US); YAJUAN WANG, YORKTOWN HEIGHTS, NY (US)

(21) Appl. No.: 14/982,830

(22) Filed: Dec. 29, 2015

(51) Int. Cl. A61B 5/00; A61B 5/145

(52) U.S. Cl. CPC A61B 5/7275; A61B 5/14532; A61B 5/7235; A61B 5/6801; A61B 5/002; A61B 5/0022

(57) ABSTRACT

A method and system for time varying risk profiling from sensor data includes receiving data time series from a plurality of sensors associated with a single patient, identifying events from the data, wherein an event is a transition between two states in the data of a sensor, formulating event prediction as a discrete state transition task using Markov jump processes to handle irregular sampling rates, estimating a transition density function for time varying continuous event probability using a hierarchical Bayesian model, and predicting risk events for the single patient by applying the hierarchical Bayesian model.

### BACKGROUND

1. Technical Field

Embodiments of the present disclosure are directed to methods and systems for time-varying risk profiling on multi-sensor health data for the prediction of continuous risk probability.

2. Discussion of the Related Art

With the increase of healthcare services in non-clinical environments using vital signs provided by wearable sensors, the desire to mine and process the physiological measurements has grown significantly. A variety of wellness management, health-monitoring and diagnosis systems have been developed, focusing on a fixed time-point events/tasks, such as stress level prediction, blood glucose level prediction, and atrial fibrillation, etc. For example, people with type 1 diabetes need to balance their desire for maintaining tight glycemic control with the risk for iatrogenic hypoglycemia. Even with recent advances in technology, hypoglycemia remains a limiting factor. Thus it is important to predict glucose values using continuous glucose monitoring (CGM) data, with the obvious application of anticipating hypoglycemia and other events, as future glucose values might be predictable using (CGM) data.

In other applications, such as mental health care, health wellness, many methods have been proposed to detect/predict risk event/outcome from sensor data. In general, data mining algorithms in these systems include the following categories: (1) Descriptive or unsupervised learning, such as clustering, association, summarization, etc.; and (2) Predictive or supervised learning, such as classification. Although many computational models have been proposed for risk event prediction/analysis, many challenges remain, such as multi-dimensionality, temporality, irregularity, bias, etc. FIG. 1 illustrates a real-world example of multi-dimensional sensor data, representing three different information streams collected from a single user, and shows the multi-dimensionality and irregularity challenges for analyzing it.

Furthermore, nearly all current methods predict risk without consideration of the time dimension. Time-varying risk profiling on multi-sensor data for the prediction of continuous risk probability over time can benefit mobile-based personal healthcare wellness applications, as well as device-based healthcare monitoring applications. The analysis framework should predict the time to a particular event, where an event here is defined as the occurrence of a specific interest point.

### SUMMARY

Exemplary embodiments of the disclosure provide systems and methods for predicting the risk probability of a time-varying event from multi-dimensional sensor data. A system according to an embodiment collects data from various sources, such as wearable sensors and from that data can predict the continuous risk probability of an event over time, rather than the interval probability on a fixed time point, and can predict multiple events simultaneously.

According to an embodiment of the disclosure, there is provided a method for time varying risk profiling from sensor data, including receiving data time series from a plurality of sensors associated with a single patient, identifying events from the data, wherein an event is a transition between two states in the data of a sensor, formulating event prediction as a discrete state transition task using Markov jump processes to handle irregular sampling rates, estimating a transition density function for time varying continuous event probability using a hierarchical Bayesian model, and predicting risk events for the single patient by applying the hierarchical Bayesian model.

According to a further embodiment of the disclosure, an event is predicted by a function q(t_{m,i}, x_{m,i}), defined by

$$q ( {t}_{m , i} , {x}_{m , i} ) = \frac{Pr ( T < ( {t}_{m , i} + Δ t ) ) - Pr ( T < {t}_{m , i} )}{1 - Pr ( T < {t}_{m , i} )} ,$$

wherein t_{m,i} is a tenure between the patient's time t_{b} in state b and the patient's time tₐ in state a associated with an i-th data observation in an m-th transition, x_{m,i} is a vector of covariates associated with the i-th data observation in the m-th transition, wherein the covariates are features associated with state a, Pr(T<t_{m,i}) is a cumulative probability of T<t_{m,i} and is given by 1−exp{−exp{βₘ^{T}x_{m,i}}t_{m,i}^{γ}^{ₘ}}, wherein (βₘ,γₘ) are parameters associated with transition m, and superscript T represents a transpose.

According to a further embodiment of the disclosure, parameters (βₘ,γₘ) are determined as those that maximize a joint likelihood function L for all variables L=p(φ)Πₘ₌₁^{M}p(βₘ,γₘ|φ)Πᵢ^{N}^{ₘ}p(t_{m,i}|βₘ,γₘ,x_{m,i}), wherein M is a number of transitions, Nₘ is a number of data observations for transition m from all users, p( ) is a probability distribution function γexp(β^{T}x)t^{γ-1}exp{−exp(β^{T} x)t^{γ}} wherein superscript T indicates a transpose, and φ=(μ_{β},Σ_{β},μ_{γ},Σ_{γ}), wherein μ_{β}, Σ_{β} are a mean and co-variance matrix for β, respectively, and μ_{γ}, Σ_{γ} are a mean and co-variance matrix for γ, respectively.

According to a further embodiment of the disclosure, the joint likelihood L is maximized by initializing parameters {μ_{β},μ_{γ}}, computing parameters (βₘ,γₘ) based on a currently value for {μ_{β},μ_{γ}} for each transition m, by gradient descent of the joint likelihood function L, and updating {μ_{β},μ_{γ}} from parameters (βₘ,γₘ) for each transition m, by gradient descent of the joint likelihood function L, wherein the steps of computing parameters (βₘ,γₘ) and updating {μ_{β},μ_{γ}} are repeated until all parameters have converged.

According to a further embodiment of the disclosure, the joint likelihood L is approximated by {c₁∥μ_{β}∥²+c₂μ_{γ}²}+Σₘ₌₁^{M}{c₃∥βₘ−μ_{β}∥²+c₄(γₘ−μ_{γ})}+Σₘ₌₁^{M}{Σₘ₌₁^{N}^{ₘ}(−log(p(t_{m,i}|βₘ,γₘ,x_{m.i})))}, wherein c₁, c₂, c₃, and c₄ are predetermined constants.

According to another embodiment of the disclosure, there is provided a method for time varying risk profiling from sensor data, including receiving a plurality of time series of events, each time series received from one of a plurality of sensors associated with a patient, determining parameters (βₘ,γₘ) of a probability distribution function p(t) of an event m occurring at time t by maximizing a joint likelihood function L=p(φ)Πₘ₌₁^{M}p(βₘ,γₘ|φ)Πᵢ^{N}^{ₘ}p(t_{m,i}|βₘ,γₘ,x_{m,i}), wherein M is a number of transitions, Nₘ is a number of data observations for transition n from all users, and φ=(μ_{β},Σ_{β},μ_{γ},Σ_{γ}), wherein μ_{β},Σ_{β} are a mean and co-variance matrix for β, respectively, μ_{γ}, Σ_{γ} are a mean and co-variance matrix for γ, respectively, t_{m,i} is a tenure between the patient's time t_{b} in state b and the patient's time tₐ in state a associated with an i-th data observation in an m-th transition, and x_{m,i} is a vector of covariates associated with the i-th data observation in the m-th transition, wherein the covariates are features associated with state a, and predicting a risk event for the patient from

$$q ( {t}_{m , i} , {x}_{m , i} ) = \frac{Pr ( T < ( {t}_{m , i} + Δ t ) ) - Pr ( T < {t}_{m , i} )}{1 - Pr ( T < {t}_{m , i} )} ,$$

wherein Pr(T<t_{m,i}) is a cumulative probability function of probability distribution function p( ) for T<t_{m,i}.

According to a further embodiment of the disclosure, the probability distribution function is p(t)=γexp(β^{T}x)t^{γ-1}exp{−exp(β^{T}x)t^{γ}}, wherein superscript T indicates a transpose.

According to a further embodiment of the disclosure, the joint likelihood L is maximized by initializing parameters {μ_{β},μ_{γ}}, computing parameters (βₘ,γₘ) based on a currently value for {μ_{β},μ_{γ}} for each transition m, and updating {μ_{β},μ_{γ}} from parameters (βₘ,γₘ) for each transition m, wherein the steps of computing parameters (βₘ,γₘ) and updating {μ_{β},μ_{γ}} are repeated until all parameters have converged.

According to a further embodiment of the disclosure, the joint likelihood L is approximated by {c₁∥μ_{β}∥²+c₂μ_{γ}²}+Σₘ₌₁^{M}{c₃∥βₘ−μ_{β}∥²+c₄(γₘ−μ_{γ})}+Σₘ₌₁^{M}{Σₘ₌₁^{N}^{ₘ}(−log(p(t_{m,i}|βₘ,γₘ,x_{m.i})))}, wherein c₁, c₂, c₃, and c₄ are predetermined constants.

According to a further embodiment of the disclosure, the events are extracted from multi-dimensional data received from the plurality of sensors, wherein events are transitions between two states in the data of a sensor.

According to a further embodiment of the disclosure, the steps of computing parameters (βₘ,γₘ) and updating {μ_{β},μ_{γ}} are performed by gradient descent of the joint likelihood function L.

According to a further embodiment of the disclosure, the data includes measurements of blood glucose levels, and the events represent changes in blood glucose levels.

According to a another embodiment of the disclosure, there is provided a non-transitory program storage device readable by a computer, tangibly embodying a program of instructions executed by the computer to perform the method steps for time varying risk profiling from sensor data.

### BRIEF DESCRIPTION OF THE DRAWINGS

FIG. 1 illustrates an example of multi-dimensional time series data, representing different information collected from a single user, according to an embodiment of the disclosure.

FIG. 2 is a flowchart of a method for time-varying risk profiling on multi-sensor data according to an embodiment of the disclosure.

FIG. 3 illustrates an example of event definition over time, according to an embodiment of the disclosure.

FIG. 4A shows a histogram of the number of observations for each event, and FIG. 4B illustrates a hierarchical model, according to an embodiment of the disclosure.

FIG. 5 is a flowchart of a maximum likelihood estimation according to an embodiment of the disclosure.

FIG. 6 is a table of statistics of measurement record durations (days) in the dataset for each of the measures for the 30 patients, according to an embodiment of the disclosure.

FIG. 7 is a table of the distributions of sampled instances, according to an embodiment of the disclosure.

FIG. 8 is a table of features derived in a time window prior to the anchored bgo state, according to an embodiment of the disclosure.

FIGS. 9A-9B illustrate prediction performance results for a fixed size prediction window, within window size of 3 hours and 6 hours, according to an embodiment of the disclosure.

FIG. 10 is a table of event prediction results for the “Normal→Hypoglycemia” event, according to an embodiment of the disclosure.

FIG. 11 is a table of event prediction results for the “Normal→Hyperglycemia” event, according to an embodiment of the disclosure.

FIG. 12 is a block diagram of an exemplary computer system for implementing a method for time-varying risk profiling on multi-sensor health data for the prediction of continuous risk probability according to an embodiment of the disclosure.

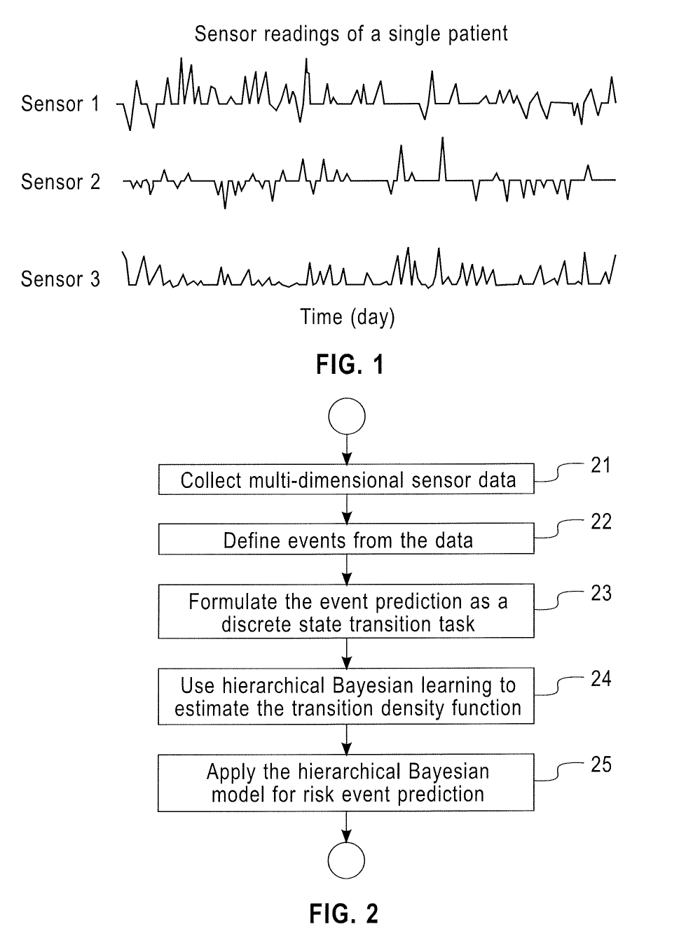

### DETAILED DESCRIPTION OF EXEMPLARY EMBODIMENTS

Exemplary embodiments of the disclosure as described herein generally include methods time-varying risk profiling on multi-sensor data for the prediction of continuous risk probability. Accordingly, while the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure. In addition, it is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

A flowchart of a method for time-varying risk profiling on multi-sensor data according to an embodiment of the disclosure is depicted in FIG. 2. Referring now to the figure, a method begins at step 21 by collecting multi-dimensional sensor data, defining or identifying events from the data at step 22, formulating the event prediction as a discrete state transition task at step 23, using a hierarchical Bayesian learning at step 24 to estimate the transition density function for time varying continuous event probability, and applying the hierarchical Bayesian model for risk event prediction, at step 25. The multi-dimensional sensor data collected at step 21 comprises data time series for a single patient collected from multiple sensors. At step 23, event predication can be formulated using Markov jump processes to handle irregular sampling rates in health sensor data. These steps will be described in detail below.

Embodiments of the disclosure consider a real-world healthcare application to demonstrate the meaning of time-to-event data. An approach according to an embodiment of the disclosure models the transformation between two successive states and related factors, and a hierarchical Bayesian framework is used to address data sparsity. A model according to an embodiment of the disclosure estimates the likelihood of a risk event as a transition of two states a certain time, which is denoted as the tenure-based risk probability. The word “tenure” means time-related or time-sensitive. This allows for predicting irregular events, how quickly/slowly the probability evolves, what the probability is as time changes, etc., rather than merely discovering patterns from the data as in an interval probability situation. A model according to an embodiment of the disclosure can handle the multi-dimensionality and irregularity challenges, the hierarchical Bayesian framework for model parameters estimation can solve the data sparsity issue for rare events, and its effectiveness has been validated on real world wearable sensor data.

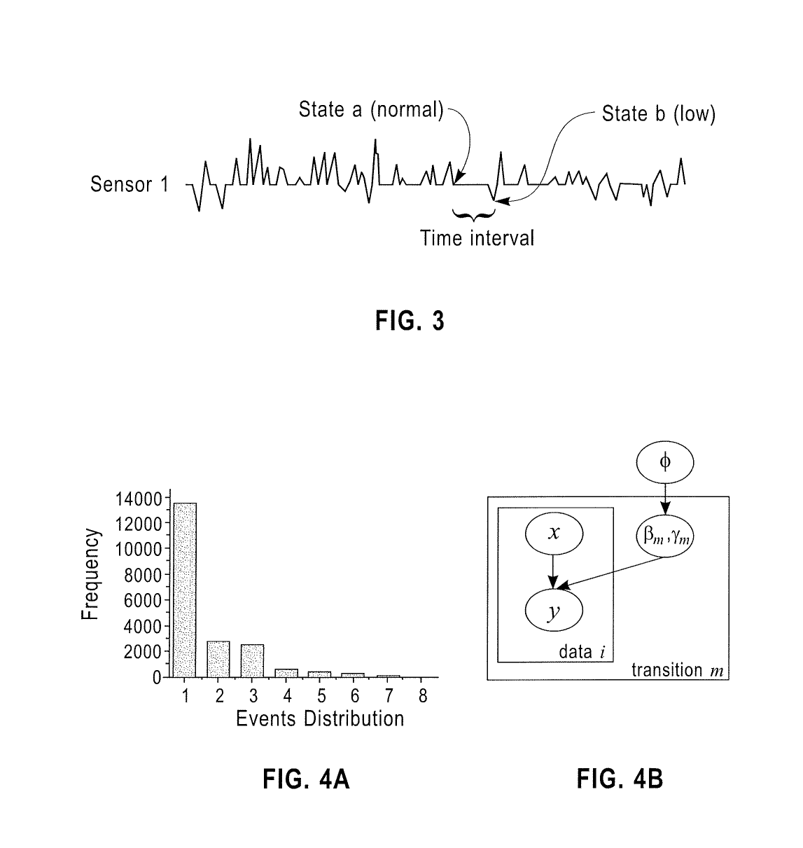

In a method according to an embodiment of the disclosure, an event can be defined as a transition between two states in the data of a sensor. FIG. 3 illustrates an example of event definition over time, according to an embodiment of the disclosure. For example, in FIG. 3, the data shown is the blood glucose value. The event “hypoglycemia” is defined as the blood glucose transfers from state a (normal) to state b (low), with an associated time interval. This transition replies on state a, state b and their associated information. A system according to an embodiment can predict different events transition probabilities with associated tenure information, under the following assumptions. The transition to the current state relies on the status of previous state, which is a Markov jump process. The state transition is not constrained, in that it can start from any state and end at any state, and the transition probability is related to the how long it takes to transition from one state to the next.

### Notations:

The following notations are used hereinbelow.
a, b=1, 2, . . . , C: The index of a status category, or state. Embodiments use a normal, high and low state.
m=1, 2, . . . , M: The index of a state transition between category a and category b.
D={D₁, . . . , Dₘ, . . . , D_{M}}: The observed data of all transitions from all patients. Note that horizontal transitions such as {a→a} is included in model according to an embodiment as well.
Dₘ={t_{m,i}, x_{m,i}}: A set of observed data associated with transition m. Each transition in has Nₘ data observations from all users. Each observation i=1, . . . , Nₘ in transition in is associated with two parts: the tenure t_{m,i} and covariates x_{m,i}.
t_{m,i}: The tenure with the i-th observation in transition m. It is the tenure between the patient's status time t_{b} and the patient's status time to at a: t_{m,i}=t_{b}−tₐ.
x_{m,i}: The k-dimensional vector of covariates associated with the i-th observation in transition m. Usually these covariates are features extracted from state a. Features can include, but are not limited to, the blood glucose value at time a, the insulin injected before time a, the carbohydrate intake value before time a, the mean blood glucose value before time a, etc.

A model according to an embodiment of the disclosure can predict the probability that a patient transits to status b at a current time, given that her/his last status a at time to and he/she did not transit up to time t_{b}.

### Risk Prediction

In survival analysis, the survival function determines the time of a particular event, such as the failure of a machine or the death of a subject. According to an embodiment, failure is considered as a transition of a patient's status to a new status. Let p(t) denote the probability density function of such an event. The cumulative distribution function P(t) and survival function S(t) are then given by:

P(y)=Pr(T≦t)

S(y)=Pr(T>t)=1−P(t) (1)

where T is a random variable denoting the survival time. In addition, the hazards function is defined as the event rate at tenure t, given that the event does not occur until tenure y or later: h(t)=p(t)S(t). In the real world, the hazards function is dependent on covariates. A classical approach incorporates covariates x in the hazards model. The Cox proportional hazards model assumes that the covariates are multiplicatively related to the hazards:

h(t)=h₀(t)exp(β^{T}x) (2)

where h₀(t) is a baseline hazards function, β is a vector of parameters, and the superscript T indicates a transpose of the vector.

According to an embodiment, the Weibull distribution is used for p(t), the probability density function of an event, which is given by

p(t)=γβt^{γ-1}exp{−βt^{γ}} (3)

for parameters γ and β. Thus, a probability density function according to an embodiment becomes:

p(t)=γexp(β^{T}x)t^{γ-1}exp{−exp(β^{T}x)t^{γ}} (4)

According to an embodiment, this probability density function represents a basic proportional hazards model that models the tenure before a transition with associated covariates, which are usually extracted from the features associated with the prediction.

According to an embodiment, the time-varying transition probability is the probability that a transition occurs at a time between t_{b} and t_{b}+Δt, given the current status at time tₐ and that the status does not change up to time t_{b}. In other words, it is the probability that the survival time T would be between t_{m,i} and t_{m,i}+Δt, given that T is not less than t_{m,i}. The prediction of a tenure-based transition probability can be denoted by q as q(t_{m,i},x_{m,i}), which is given by:

$$\begin{matrix}\begin{matrix}q ( {t}_{m , i} , {x}_{m , i} ) = Pr ( {t}_{m , i} < T \leq {t}_{m , i} + Δ t \vert T > {t}_{m , i} ) \\ = \frac{Pr ( T < ( {t}_{m , i} + Δ t ) ) - Pr ( T < {t}_{m , i} )}{1 - Pr ( T < {t}_{m , i} )} ,\end{matrix} \\ where & ( 5 ) \\ Pr ( T < {t}_{m , i} ) = 1 - exp \{ - exp \{ {β}_{m}^{T} {x}_{m , i} \} {t}_{m , i}^{{γ}_{m}} \} . & ( 6 )\end{matrix}$$

Each transition m, corresponding to each defined event, has its own parameters (βₘ,γₘ). A learning task according to an embodiment is to infer all parameters {(βₘ,γₘ)}ₘ₌₁^{M} from training data.
Extension with Bayesian Framework

A straightforward way to learn the parameters is to learn them in parallel. However, in real use cases, the number of samples for each event tends to follow a power law distribution. FIG. 4A shows a histogram of the number of observations for each event, according to an embodiment of the disclosure. A few samples are frequently observed while most events are rare, and the event distribution is heavily unbalanced, making it challenging to learn parameters of the corresponding hazards model. According to an embodiment of the disclosure, to address the data sparsity issue, a proportional hazards model can be extended with a hierarchical Bayesian framework. A hierarchical Bayesian framework according to an embodiment can borrow information from other transitions when learning the parameters for transition m. FIG. 4B illustrates a hierarchical model in a hierarchical proportional hazards model according to an embodiment, which shows dependencies of variables βₘ, γₘ on a prior φ, and the effect of the variables on the transition in from state x to state y triggered by data i. Models of each transition share information through the prior, φ=(μ_{β},Σ_{β},μ_{γ},Σ_{γ}), where μ_{β}, Σ_{β} are the mean and co-variance matrix for β, respectively, and μ_{γ}, Σ_{γ} are the mean and co-variance matrix for γ, respectively. Let θ={φ, β₁, λ₁, . . . , β_{M}, λ_{M}} represent parameters that need to be estimated. A joint likelihood for all variables in a probabilistic model according to an embodiment is:

L(D,θ)=p(φ)Πₘ₌₁^{M}p(βₘ,γₘ|φ)Πᵢ^{N}^{ₘ}p(t_{m,i}|βₘ,γₘ,x_{m,i}), (7)

where the probability density function p( ) is the Weibull distribution disclosed above.

### Parameter Estimation

A model according to an embodiment of the disclosure contains many hidden variables, some of which are high-dimensional vectors. Hence, a traditional Bayesian method may be too computationally expensive to learn the model. Instead, according to an embodiment of the disclosure, an iterative method is used with a point estimation at each step. According to an embodiment, constants cᵢ, i=1, 2, 3, 4, can replace functions of μ_{β}, Σ_{β}, μ_{γ}, Σ_{γ}, respectively, with the same model effect. These constants can be set by cross-validation in experiments, described below. The constants can be viewed as regularization factors to avoid overfitting and can be set by cross-validation in an experiment. A maximum likelihood estimation according to an embodiment of the remaining parameters is given by EQ. (8).

$$( {μ}_{β} , {μ}_{γ} , {β}_{1} , {γ}_{1} , … , {β}_{M} , {γ}_{M} ) = argmax L ( D , θ ) = argmin \{ {c}_{1} {‖ {μ}_{β} ‖}^{2} + {c}_{2} {μ}_{γ}^{2} \} + {\sum }_{m = 1}^{M} \{ {c}_{3} {‖ {β}_{m} - {μ}_{β} ‖}^{2} + {c}_{4} ( {γ}_{m} - {μ}_{γ} ) \} + {\sum }_{m = 1}^{M} \{ {\sum }_{m = 1}^{{N}_{m}} ( - log ( p ( {t}_{m , i} \vert {β}_{m} , {γ}_{m} , {x}_{m , i} ) ) ) \} \qquad ( 8 )$$

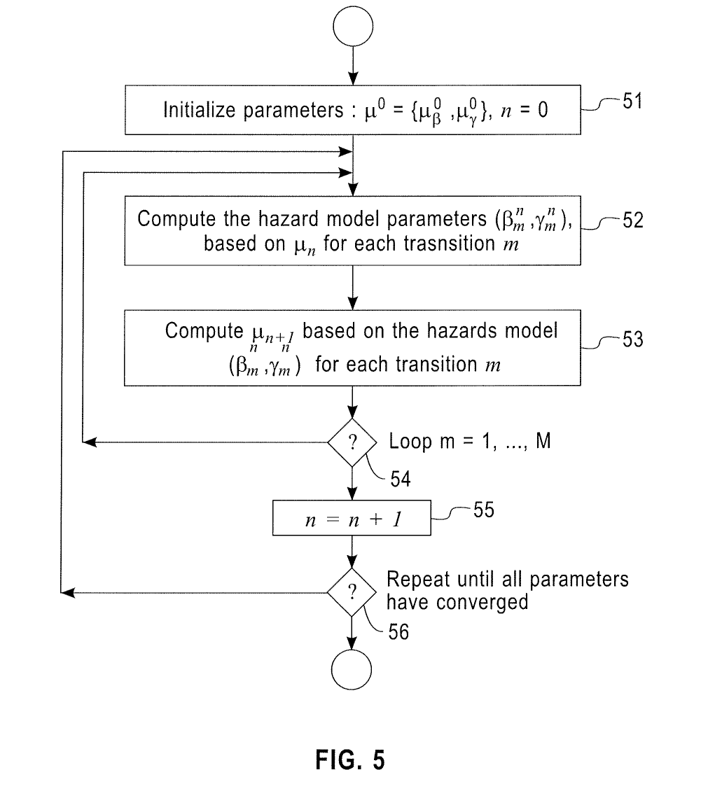

According to an embodiment of the disclosure, a method to solve the previous equation is shown in Algorithm 1, also illustrated in the flowchart of FIG. 5. A method according to an embodiment first initializes μ₀ and then updates parameters by iteratively performing steps 3-7 until convergence.

Algorithm 1 Hierarchical Bayesian framework for parameters learning.
1: Initialization: μ⁰={μ_{β}⁰,μ_{γ}⁰}, n=0 (Step 51)

### 2: Repeat

3: for m=1 to M:
4: Compute hazard model parameters (βₘⁿ,γₘⁿ) using Eq. (8), based on μⁿ for each transition m (Step 52)
5: Compute μⁿ⁺¹ using Eq. (8) based on hazards model (βₘⁿ,γₘⁿ) for each transition m (Step 53)

### 6: end for (Step 54)

7: n=n+1 (Step 55)
8: until all parameters have converged (Step 56)
9: return
According to an embodiment, steps 4 and 5 can be performed with a conjugate gradient descent.

### Evaluation

A framework according to an embodiment of the disclosure can be applied to real clinical cohorts to demonstrate predictive performance of a method according to an embodiment.

### Experimental Setting

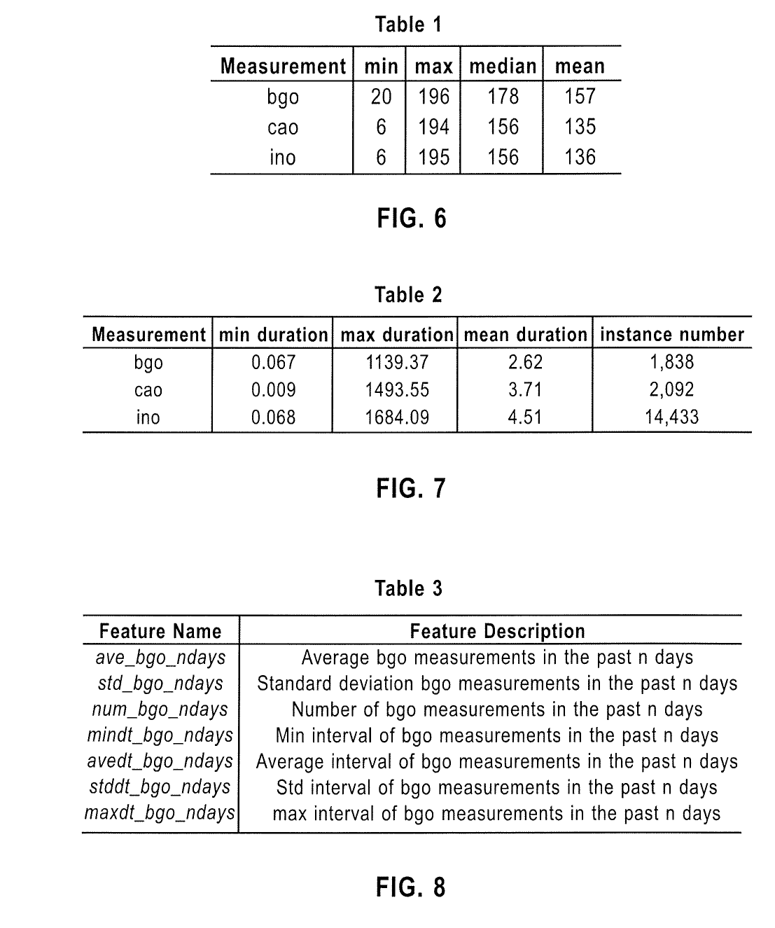

A dataset according to an embodiment is composed by measures of blood glucose level (bgo-mg/dl), carbohydrate intake (cao-grams), and insulin injected (ino-units) from self-monitored type 1 diabetes patients. In total, there are 30 patients. The statistics of measurement record durations (days) in the dataset for each of the measures for the 30 patients are listed in Table 1, shown in FIG. 6. The duration of records varies from 6 days to 6 months for an individual patient.

A risk prediction task according to an embodiment of hypoglycemia and hyperglycemia events in self-monitored type 1 diabetes patients is framed as a detection of the probability of bgo change from a current normal state (72 mg/dl<bgo<270 mg/dl) to either hypoglycemia (bgo<72 mg/dl) or hyperglycemia (bgo>270 mg/dl) state. The original input data is organized to support the prediction of the following state transitions, which maximizes the utility of available data and handles the irregular measurement rates. In the longitudinal records, three adjacent state transition pairs were identified as follows, which served as the anchors to form training and testing data instances in this study.

According to a method according to an embodiment, all pair-wise patterns need to be extracted for the three different events, which cover the two successive points in the sensor as well as their related information. Table 2, shown in FIG. 7, shows the distributions of sampled instances. As can be seen from the table, the sampling frequency is highly uneven. There are 14,433 instances for “Normal→Normal” but only 2,092 for “Normal→Hyperglycemia” and 1,838 for “Normal→Hypoglycemia”.

For each of data instance mentioned in the above section, features were derived in the time-domain anchored by the time stamp of current normal blood glucose state. The statistics of each of the three measures in the past n days were derived. Note that features were extracted based on different time window (n=1, 3, 7). Here, the feature description is shown when n=l and the result shown later is also based on the feature with window n=1 day. Features were also derived based on different states. Table 3, shown in FIG. 8, shows features derived in a time window prior to the anchored bgo state, which is similar for ino and cao used in this study. According to an embodiment, the following models were implemented as baselines: (1) prediction with random forest; (2) prediction with logistic regression; and (3) Cox proportional hazards model (Cox). It is noticeable that among all the baselines, only Cox can provide risk prediction as a function of time. For comparison purpose, regular prediction is performed and the performance is measured with AUC. The samples are randomly split with the ratio of training vs. testing being 9:1. For the sake of fairness, the splitting is the same for all methods in each iteration.

### Performance Evaluation

Prediction with Fixed Size Window:

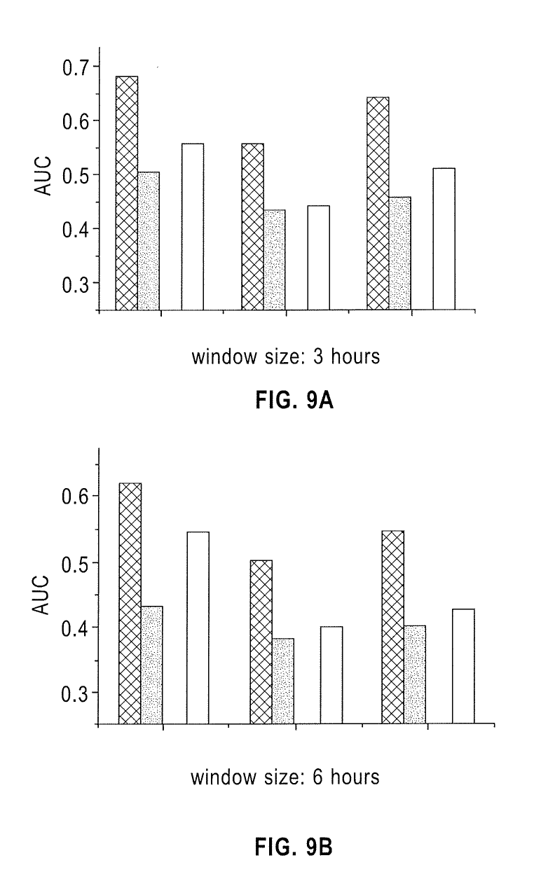

Prediction performance results for a fixed size prediction window are summarized in FIGS. 9A-9B, within window size of 3 hours and 6 hours. In FIG. 9A-9B, the left group of bars represents the Normal→Hyperglycemia event, the center group of bars represents the Normal→Hypoglycemia event, and the right group opf bars represents the Normal→Normal event. In each group of bars, the bars represent, from left to right, a result of a risk profiling according to an embodiment, the results of a Cox proportional hazards model, the result from a random forest model, and the result from a logistic regression. As can be seen from the figures, a method according to an embodiment of the disclosure outperforms the other baselines. Measured by AUC, a method according to an embodiment increases the AUC prediction by more than 7.3% for the “Normal→Hyperglycemia” event, 6.4% for the “Normal→Hypoglycemia” event and 9.0% for the “Normal→Normal” event, respectively. The random forest and logistic regression models have comparable performance as their architectures are similar. The prediction accuracy of the Cox model is worse than random forest and logistic regression. One possible reason might be that the Cox model cannot learn well with such unbalanced classes.

### Prediction for the Next Event:

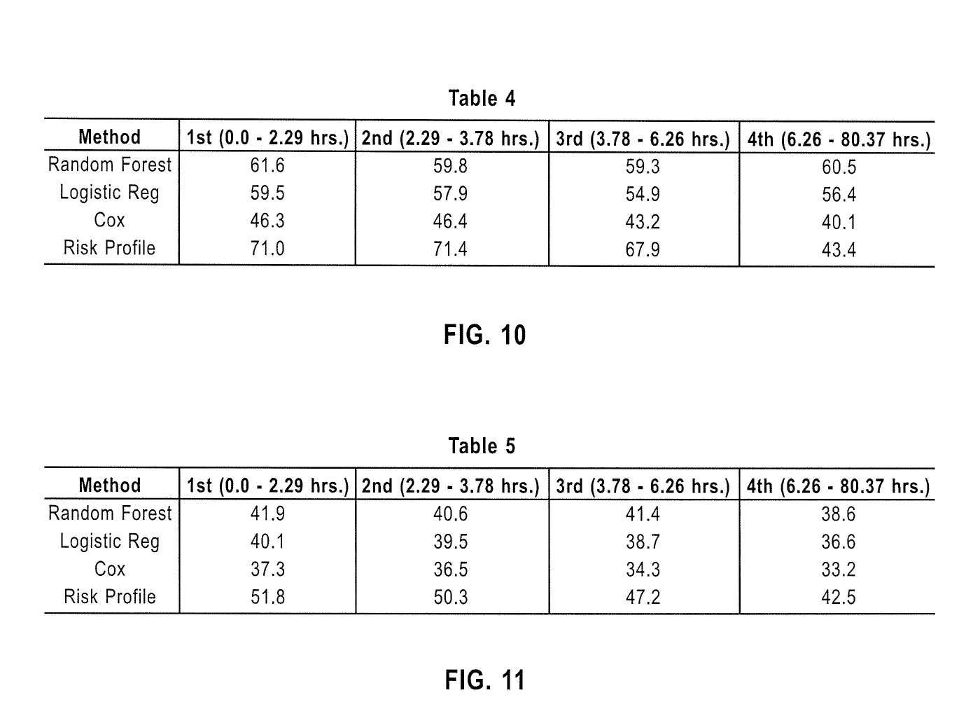

Next event prediction results for the “Normal→Hypoglycemia” event is shown in Table 4, shown in FIG. 10, which summarizes the AUC into four time duration ranges: 1st (0.0-2.29 hrs.), 2nd (2.29-3.78 hrs), 3rd (3.78-6.26 hrs) and 4th (6.26-80.37 hrs). A method according to an embodiment outperforms all the baselines. Similar trends can be observed for the event “Normal→Hyperglycemia” shown in Table 5, shown in FIG. 11. It is notable that with the time duration decreases, the performance gains of all proposed models also increase, which shows that a model according to an embodiment would also benefit if the prediction is small.

In summary, the experimental results have demonstrated the effectiveness of a model according to an embodiment on real wearable sensors data. A risk profiling framework according to an embodiment can improve predictive performance, which shows that incorporating temporal connectivity can boost performance.

### System Implementations

As will be appreciated by one skilled in the art, embodiments of the present disclosure may be embodied as a system, method or computer program product. Accordingly, embodiments of the present disclosure may take the form of an entirely hardware embodiment, an entirely software embodiment (including firmware, resident software, micro-code, etc.) or an embodiment combining software and hardware embodiments that may all generally be referred to herein as a “circuit,” “module” or “system.” Furthermore, embodiments of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Embodiments of the present disclosure are described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

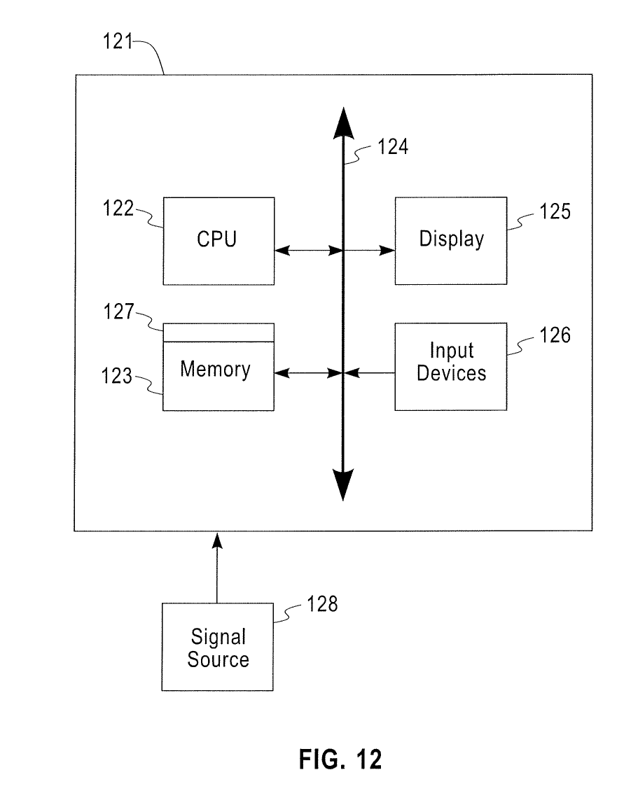

FIG. 12 is a block diagram of an exemplary computer system for implementing a method for time-varying risk profiling on multi-sensor health data according to an embodiment of the disclosure. Referring now to FIG. 12, a computer system 121 for implementing the present disclosure can comprise, inter alia, a central processing unit (CPU) 122, a memory 123 and an input/output (I/O) interface 124. The computer system 121 is generally coupled through the I/O interface 124 to a display 125 and various input devices 126 such as a mouse and a keyboard. The support circuits can include circuits such as cache, power supplies, clock circuits, and a communication bus. The memory 123 can include random access memory (RAM), read only memory (ROM), disk drive, tape drive, etc., or a combinations thereof. The present disclosure can be implemented as a routine 127 that is stored in memory 123 and executed by the CPU 122 to process the signal from the signal source 128. As such, the computer system 121 is a general purpose computer system that becomes a specific purpose computer system when executing the routine 127 of the present disclosure.

The computer system 121 also includes an operating system and micro instruction code. The various processes and functions described herein can either be part of the micro instruction code or part of the application program (or combination thereof) which is executed via the operating system. In addition, various other peripheral devices can be connected to the computer platform such as an additional data storage device and a printing device.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the present disclosure has been described in detail with reference to exemplary embodiments, those skilled in the art will appreciate that various modifications and substitutions can be made thereto without departing from the spirit and scope of the disclosure as set forth in the appended claims.

What is claimed is:

1. A method for time varying risk profiling from sensor data, comprising the steps of:
receiving data time series from a plurality of sensors associated with a single patient;
identifying events from the data, wherein an event is a transition between two states in the data of a sensor;
formulating event prediction as a discrete state transition task using Markov jump processes to handle irregular sampling rates;
estimating a transition density function for time varying continuous event probability using a hierarchical Bayesian model; and
predicting risk events for the single patient by applying the hierarchical Bayesian model.

2. The method of claim 1, wherein an event is predicted by a function q(t_{m,i}, x_{m,i}), defined by
$$q ( {t}_{m , i} , {x}_{m , i} ) = \frac{Pr ( T < ( {t}_{m , i} + Δ t ) ) - Pr ( T < {t}_{m , i} )}{1 - Pr ( T < {t}_{m , i} )} ,$$
wherein
t_{m,i} is a tenure between the patient's time t_{b} in state b and the patient's time tₐ in state a associated with an i-th data observation in an m-th transition,
x_{m,i} is a vector of covariates associated with the i-th data observation in the m-th transition, wherein the covariates are features associated with state a,
Pr(T<t_{m,i}) is a cumulative probability of T<t_{m,i} and is given by 1−exp{−exp{βₘ^{T}x_{m,i}}t_{m,i}^{γ}^{ₘ}}, wherein (βₘ,γₘ) are parameters associated with transition m, and superscript T represents a transpose.

3. The method of claim 2, wherein parameters (βₘ,γₘ) are determined as those that maximize a joint likelihood function L for all variables L=p(φ)Πₘ₌₁^{M}p(βₘ,γₘ|φ)Πᵢ^{N}^{ₘ}p(t_{m,i}|βₘ,γₘ,x_{m,i}), wherein M is a number of transitions, Nₘ is a number of data observations for transition m from all users, p( ) is a probability distribution function γexp(β^{T}x)t^{γ-1}exp{−exp(β^{T}x)t^{γ}} wherein superscript T indicates a transpose, and φ=(μ_{β},Σ_{β},μ_{γ},Σ_{γ}), wherein μ_{β}, Σ_{β} are a mean and co-variance matrix for β, respectively, and μ_{γ}, Σ_{γ} are a mean and co-variance matrix for γ, respectively.

4. The method of claim 3, wherein the joint likelihood L is maximized by
initializing parameters {μ_{β},μ_{γ}},
computing parameters (βₘ,γₘ) based on a currently value for {μ_{β},μ_{γ}} for each transition m, by gradient descent of the joint likelihood function L, and
updating {μ_{β},μ_{γ}} from parameters (βₘ,γₘ) for each transition m, by gradient descent of the joint likelihood function L,
wherein the steps of computing parameters (βₘ,γₘ) and updating {μ_{β},μ_{γ}} are repeated until all parameters have converged.

5. The method of claim 4, wherein the joint likelihood L is approximated by
$$\{ {c}_{1} {‖ {μ}_{β} ‖}^{2} + {c}_{2} {μ}_{γ}^{2} \} + {\sum }_{m = 1}^{M} \{ {c}_{3} {‖ {β}_{m} - {μ}_{β} ‖}^{2} + {c}_{4} ( {γ}_{m} - {μ}_{γ} ) \} + {\sum }_{m = 1}^{M} \{ {\sum }_{m = 1}^{{N}_{m}} ( - log ( p ( {t}_{m , i} \vert {β}_{m} , {γ}_{m} , {x}_{m , i} ) ) ) \} ,$$
wherein c₁, c₂, c₃, and c₄ are predetermined constants.

6. A method for time varying risk profiling from sensor data, comprising the steps of:
receiving a plurality of time series of events, each time series received from one of a plurality of sensors associated with a patient;
determining parameters (βₘ,γₘ) of a probability distribution function p(t) of an event m occurring at time t by maximizing a joint likelihood function L=p(φ)Πₘ₌₁^{M}p(βₘ,γₘ|φ)Πᵢ^{N}^{ₘ}p(t_{m,i}|βₘ,γₘ,x_{m,i}), wherein M is a number of transitions, Nₘ is a number of data observations for transition in from all users, and φ=(μ_{β},Σ_{β},μ_{γ},Σ_{γ}), wherein μ_{β}, Σ_{β} are a mean and co-variance matrix for β, respectively, μ_{γ}, Σ_{γ} are a mean and co-variance matrix for γ, respectively, t_{m,i} is a tenure between the patient's time t_{b} in state b and the patient's time tₐ in state a associated with an i-th data observation in an m-th transition, and x_{m,i} is a vector of covariates associated with the i-th data observation in the m-th transition, wherein the covariates are features associated with state a; and
predicting a risk event for the patient from
$$q ( {t}_{m , i} , {x}_{m , i} ) = \frac{Pr ( T < ( {t}_{m , i} + Δ t ) ) - Pr ( T < {t}_{m , i} )}{1 - Pr ( T < {t}_{m , i} )} ,$$
wherein Pr(T<t_{m,i}) is a cumulative probability function of probability distribution function p( ) for T<t_{m,i}.

7. The method of claim 6, wherein the probability distribution function is p(t)=γexp(β^{T}x)t^{γ-1}exp{−exp(β^{T}x)t^{γ}}, wherein superscript T indicates a transpose.

8. The method of claim 6, wherein the joint likelihood L is maximized by
initializing parameters {μ_{β},μ_{γ}},
computing parameters (βₘ,γₘ) based on a currently value for {μ_{β},μ_{γ}} for each transition m, and
updating {μ_{β},μ_{γ}} from parameters (βₘ,γₘ) for each transition m,
wherein the steps of computing parameters (βₘ,γₘ) and updating {μ_{β},μ_{γ}} are repeated until all parameters have converged.

9. The method of claim 8, wherein the joint likelihood L is approximated by
{c₁∥μ_{β}∥²+c₂μ_{γ}²}+Σₘ₌₁^{M}{c₃∥βₘ−μ_{β}∥²+c₄(γₘ−μ_{γ})}+Σₘ₌₁^{M}{Σₘ₌₁^{N}^{ₘ}(−log(p(t_{m,i}|βₘ,γₘ,x_{m.i})))},
wherein c₁, c₂, c₃, and c₄ are predetermined constants.

10. The method of claim 6, wherein the events are extracted from multi-dimensional data received from the plurality of sensors, wherein events are transitions between two states in the data of a sensor.

11. The method of claim 8, wherein the steps of computing parameters (βₘ,γₘ) and updating {μ_{β},μ_{γ}} are performed by gradient descent of the joint likelihood function L.

12. The method of claim 10, wherein the data includes measurements of blood glucose levels, and the events represent changes in blood glucose levels.

13. A non-transitory program storage device readable by a computer, tangibly embodying a program of instructions executed by the computer to perform the method steps for time varying risk profiling from sensor data, the method comprising the steps of:
receiving a plurality of time series of events, each time series received from one of a plurality of sensors associated with a patient;
determining parameters (βₘ,γₘ) of a probability distribution function p(t) of an event m occurring at time t by maximizing a joint likelihood function L=p(φ)Πₘ₌₁^{M}p(βₘ,γₘ|φ)Πᵢ^{N}^{ₘ}p(t_{m,i}|βₘ,γₘ,x_{m,i}), wherein M is a number of transitions, Nₘ is a number of data observations for transition m from all users, and φ=(μ_{β},Σ_{β},μ_{γ},Σ_{γ}), wherein μ_{β}, Σ_{β} are a mean and co-variance matrix for β, respectively, μ_{γ}, Σ_{γ} are a mean and co-variance matrix for γ, respectively, t_{m,i} is a tenure between the patient's time t_{b} in state b and the patient's time tₐ in state a associated with an i-th data observation in an m-th transition, and x_{m,i} is a vector of covariates associated with the i-th data observation in the m-th transition, wherein the covariates are features associated with state a; and
predicting a risk event for the patient from
$$q ( {t}_{m , i} , {x}_{m , i} ) = \frac{Pr ( T < ( {t}_{m , i} + Δ t ) ) - Pr ( T < {t}_{m , i} )}{1 - Pr ( T < {t}_{m , i} )} ,$$
wherein Pr(T<t_{m,i}) is a cumulative probability function of probability distribution function p( ) for T<_{m,i}.

14. The computer readable program storage device of claim 13, wherein the probability distribution function is p(t)=γexp(β^{T}x)t^{γ-1}exp{−exp(β^{T}x)t^{γ}}, wherein superscript T indicates a transpose.

15. The computer readable program storage device of claim 13, wherein the joint likelihood L is maximized by
initializing parameters {μ_{β},μ_{γ}},
computing parameters (βₘ,γₘ) based on a currently value for {μ_{β},μ_{γ}} for each transition m, and
updating {μ_{β},μ_{γ}} from parameters (βₘ,γₘ) for each transition m,
wherein the steps of computing parameters (βₘ,γₘ) and updating {μ_{β},μ_{γ}} are repeated until all parameters have converged.

16. The computer readable program storage device of claim 15, wherein the joint likelihood L is approximated by
{c₁∥μ_{β}∥²+c₂μ_{γ}²}+Σₘ₌₁^{M}{c₃∥βₘ−μ_{β}∥²+c₄(γₘ−μ_{γ})}+Σₘ₌₁^{M}{Σₘ₌₁^{N}^{ₘ}(−log(p(t_{m,i}|βₘ,γₘ,x_{m.i})))},
wherein c₁, c₂, c₃, and c₄ are predetermined constants.

17. The computer readable program storage device of claim 13, wherein the events are extracted from multi-dimensional data received from the plurality of sensors, wherein events are transitions between two states in the data of a sensor.

18. The computer readable program storage device of claim 15, wherein the steps of computing parameters (βₘ,γₘ) and updating {μ_{β},μ_{γ}} are performed by gradient descent of the joint likelihood function L.

19. The computer readable program storage device of claim 17, wherein the data includes measurements of blood glucose levels, and the events represent changes in blood glucose levels.
